(19) United States
(12) Patent Application Publication
(10) Pub. No.: US 2019/0246942 A1
(43) Pub. Date: Aug. 15, 2019

(54) Body Composition Analysis Apparatus

(71) Applicant: TomTom International B.V., Amsterdam (NL)

(72) Inventors: Anastasia Moreva, Amsterdam (NL); Brie Betz, Amsterdam (NL); Walter Hermsen, Amsterdam (NL); Ali Erdi Tugmaner, Amsterdam (NL); Nevil Sreedhar, Amsterdam (NL); Borja Fernandez Rodriguez, Amsterdam (NL)

(21) Appl. No.: 16/329,408

(22) PCT Filed: Aug. 31, 2017
(86) PCT No.: PCT/EP2017/071918

§ 371 Date: Feb. 28, 2019

(30) Foreign Application Priority Data
Sep. 1, 2016 (GB) 1614885.0

(51) Int. Cl. A61B 5/053; A61B 5/11; A61B 5/00

(52) U.S. Cl. CPC A61B 5/0537; A61B 5/4872; A61B 5/681; A61B 5/1118

(57) ABSTRACT

A wearable device for performing a plurality of functions including a first function to measure a body composition parameter of a user wearing the device and one or more second functions requiring an input from the user. The device comprises a first electrode arranged, when the device is worn by a user, to contact the body of the user, a second electrode arranged to be touched by the user, a touch controller arranged to detect when a user touches the second electrode, a body composition parameter measurement device arranged, when the user is in contact with the second electrode, to measure a body impedance of the user by passing a current between the first and second electrodes and detecting a voltage generated between the first and second electrode in response to the current, and to use the measured body impedance to determine a body composition parameter.

### FIELD OF INVENTION

The present invention is concerned with providing a measure or indication of body composition of a user, in particular a measure of body fat and/or muscle.

### BACKGROUND OF THE INVENTION

In recent times there have been great developments in the fields of health and fitness. In general, more people are concerned with living a healthy life and are concerned to keep fit and healthy. With improvements and advancements in available information technology, more information on health and fitness is available to users. Fitness magazines and online sources enable people to be kept up-to-date on the latest medical knowledge and technical advances that can help them maintain a healthy lifestyle. New devices make it easy for people to track their fitness and/or aspects of their life and physical or physiological parameters in their quest to remain or become fit and healthy.

For example, tools are available to enable people to: calculate their body mass index (BMI) and to compare this to healthy values; count amounts of activity, sleep, calories consumed/expended and heart rate and compare these to healthy values; determine blood sugar levels, cholesterol values, etc; and measure parameters, such as impedance, which can be used, for example, to analyse their body composition, e.g. levels of body fat. Devices have developed in line with the available information and the user's desire to identify their own fitness levels.

In addition to access to information and tools via the Internet or the like, there are now many devices on the market that enable a user to track their fitness in a simple and convenient way, such as apps on mobile telephones and wearable fitness trackers, such as wrist-worn devices and watches incorporating tracking, measuring and sensing functions.

Bio-impedance analysis (BIA) is a technique for measuring body composition, e.g. fat, muscle, etc based on user inputs including impedance. BIA determines the electrical impedance provided by the user's body tissue which can then derive a ratio of body fat to body moisture. Simple devices are known for measuring body fat using BIA using electrodes attached to parts of a user's body; such devices have been found to be not sufficiently accurate for absolute one-off measurements but are useful for tracking changes in an individual over time. The accuracy of the readings is, however, affected by a number of factors and can also vary considerably, for any particular user, over the course of a day due to, for example, times when meals are consumed, hydration at any time, and also the location of the measuring electrodes on the user.

Simple devices for measuring body impedance include two electrodes placed on, e.g. the user's two feet; more accurate results have been found using four electrodes on the hands and feet, i.e. two current and voltage electrodes for each hand and foot, or even more electrodes on the user's body.

An impedance measurement circuit comprises a current source, a voltage measurement circuit and a processor. Impedance can be determined using two sensors—a so-called ‘two-point’ system—whereby current from the source is passed through the body whose impedance is to be measured, from one electrode in contact with the body at one location to a second electrode in contact with the body at another location. The voltage measurement circuit measures the voltage drop across the electrodes to determine the impedance.

Accuracy of the impedance measurement can be improved using a ‘four-point’ system which uses an additional pair of electrodes. Current is fed through two ‘feeding’ electrodes and the voltage drop is measured between two ‘measurement’ electrodes. An example of such an impedance measurement can be found in US 2011/0208458 A1.

As mentioned above, there is an increased demand for wearable or easily portable health and fitness monitoring devices. Such four electrode hand/foot devices do not easily lend themselves to a wearable form. In recent times, algorithms and devices have been developed to add BIA analysis and other body parameter measurement and analysis functions to wearable devices such as wrist-worn fitness trackers. One such device and algorithm is taught in US 2016/089053 A1. Impedance is measured using two electrodes or two pairs of electrodes, one in contact with the user's wrist and another on the outer-facing side of the device which the user touches, e.g. with a finger. Touching the outer electrode completes the circuit from the electrode touching the user's wrist to enable a body impedance measurement. A similar device is taught in US 2016/0106337 A1, which uses both two-point and four-point measurements.

As mentioned above, wearable devices have become very popular and there is a desire to add more functions and capabilities to such devices without affecting their usability, i.e. without them having to be too large to be worn comfortably and without adversely affecting the aesthetic appearance of the device.

Such devices usually have a processor configured to provide various functions and operating and/or display modes and control buttons/touch sensor inputs to provide input to the processor to, for example, switch between modes, wake up the device, input settings, start and stop activities, etc. Many devices such as, but not exclusively, BIA devices also have, as discussed above, a sensor acting as an electrodes in a measurement/analysis circuit, which has to be touched by the user to complete the measurement circuit and is, therefore, on the outer side of the device when worn. It would be desirable to increase the functionality/capability of this sensor.

### SUMMARY OF THE INVENTION

According to an aspect of the invention, there is provided a wearable device configured to perform a plurality of functions including a first function to measure a body composition parameter of a user wearing the device and one or more second functions requiring an input from the user, the device comprising:

a first electrode on an inner surface of the device arranged, when the device is worn by a user, to contact the body of the user;

a second electrode on an outer surface of the device arranged to be touched by the user;

a touch controller arranged to detect when a user touches the second electrode;

a body composition parameter measurement device arranged, when the user is in contact with the second electrode, to measure a body impedance of the user by passing a current between the first and second electrodes and detecting a voltage generated between the first and second electrode in response to the current, and to use the measured body impedance to determine a value of a body composition parameter;

a processor arranged to control a switch that, when in a first state, electrically connects the second electrode with the touch controller, such that a detected touch by the user on the second electrode can be used as an input to control a second function of the device, and, when in a second state, electrically connects the second electrode electrode with the body composition parameter measurement device; and

an input device arranged to receive an input from the user to select one of the plurality of functions, and to provide an indication of said selected function to the processor,

wherein the processor is further arranged to change the state of the switch from the first state to the second state when the input device indicates that the selected function is the first function.

In a preferred embodiment, the input device comprises a touch screen display device, and the user preferably selects a function of the device by scrolling or navigating a displayed menu.

The one or more second functions of the device that require an input from the user can comprise one or more of the following: to wake up the device from a low power state, e.g. by activating the input device, e.g. touch screen display device; to start and/or stop a timer or stop watch; and to indicate the beginning and/or end of an activity.

The plurality of functions that the device can perform preferably includes one or more third functions that do not require an input from the user, e.g. they are passive functions, such as providing information to a user using an output device. The output device can be the same as the input device when, for example, the input device is a touch screen display. The one or more third functions of the device can comprise one or more of the following: to provide a current heart rate of the user, e.g. as measured using an optical heart rate sensor on the inner surface of the device; to provide the current time to the user; and to provide a set of metrics associated with a previously competed activity, such as distance travelled, time elapsed, etc.

The body composition parameter is preferably at least one of body fat percentage and body muscle percentage.

In embodiments, the device is arranged to be worn on a wrist of the user, such the first electrode would be in contact with the wrist, and the second electrode would typically come into contact with a finger of the user. The device preferably therefore further comprises a strap to allow the device to be secured to the user's wrist. The strap can be integral with a main body of the device (that includes the components of the invention described above). Alternatively, the body of the device can be removably connected to the strap.

The second electrode is preferably shaped, so as to facilitate contact with the user's finger when it is touched. Therefore, for example, the second electrode is curved, i.e. has a convex surface geometry.

The present invention in accordance with any of its further aspects or embodiments may include any of the features described in reference to other aspects or embodiments of the invention to the extent it is not mutually inconsistent therewith.

### BRIEF DESCRIPTION OF THE FIGURES

Various embodiments will now be described, by way of example only, and with reference to the accompanying drawings in which:

FIG. 1A is a perspective view of a wrist-worn activity tracker that can incorporate the invention;

FIG. 1B is an alternative perspective view of the activity tracker of FIG. 1A shown the inside or skin-facing side of the device;

FIG. 2 is a schematic diagram of the various features and components that can be provided in an activity or fitness tracker;

FIG. 3 is a schematic diagram showing the electrical connections between the electrodes of a wrist-worn activity tracker;

FIG. 4 shows an exemplary menu structure used in an activity or fitness tracker;

FIG. 5 shows exemplary feedback that can be provided to user following a body composition parameter measurement; and

FIG. 6 shows an exemplary navigation flow associated with monitoring an activity using an activity or fitness tracker.

### DETAILED DESCRIPTION OF THE FIGURES

The embodiments below relate to the invention incorporated in a wrist-worn or other wearable device such as a sports watch, or activity or fitness tracker. The invention can, however, be incorporated in other devices such as another mobile device, such as a mobile phone, or on a web server receiving the data values from another device such as those listed here.

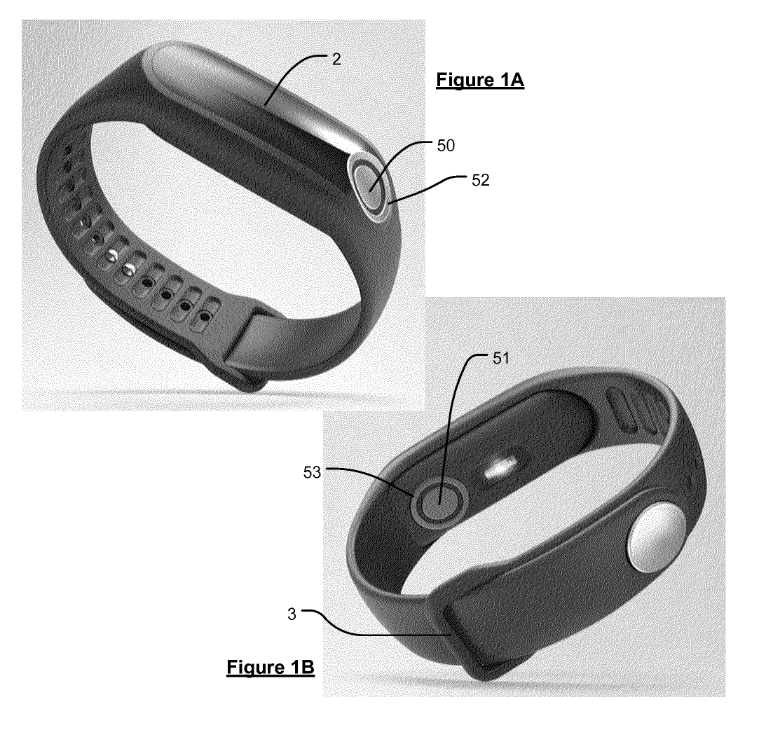

Referring to FIGS. 1A and 1B, the invention may be incorporated in a wrist-worn tracker comprising a wrist strap 1 and a tracker module 2 attached to, fitted into, mounted on or in or detachably mounted to the strap 1. The strap may be an elastic or stretchable strap or may be an adjustable strap with a fastener/buckle 3.

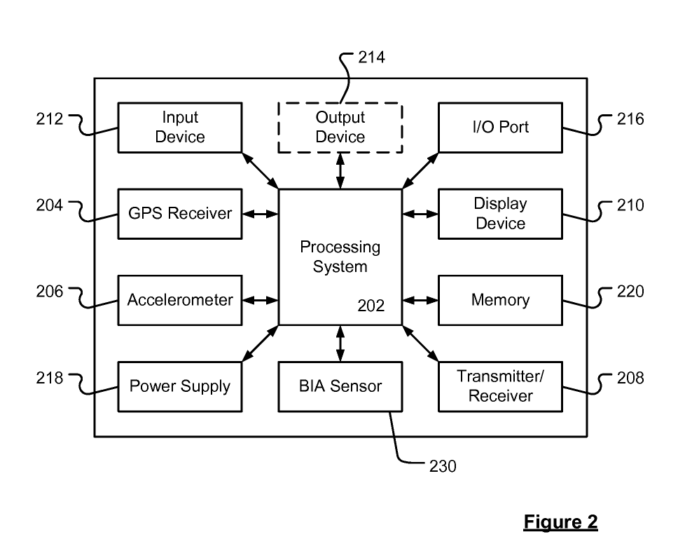

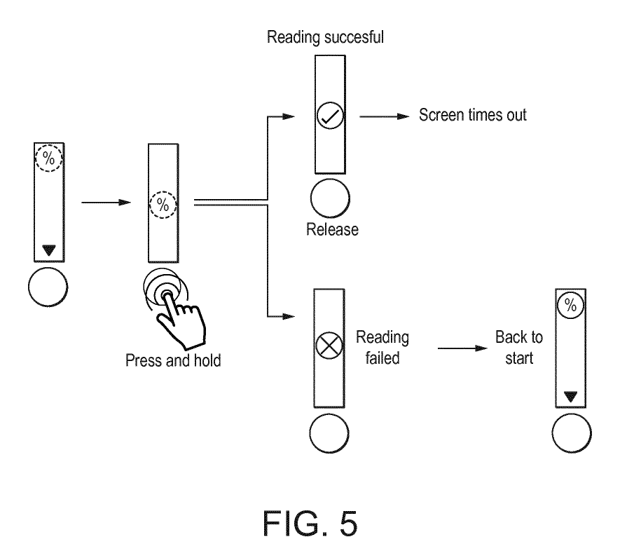

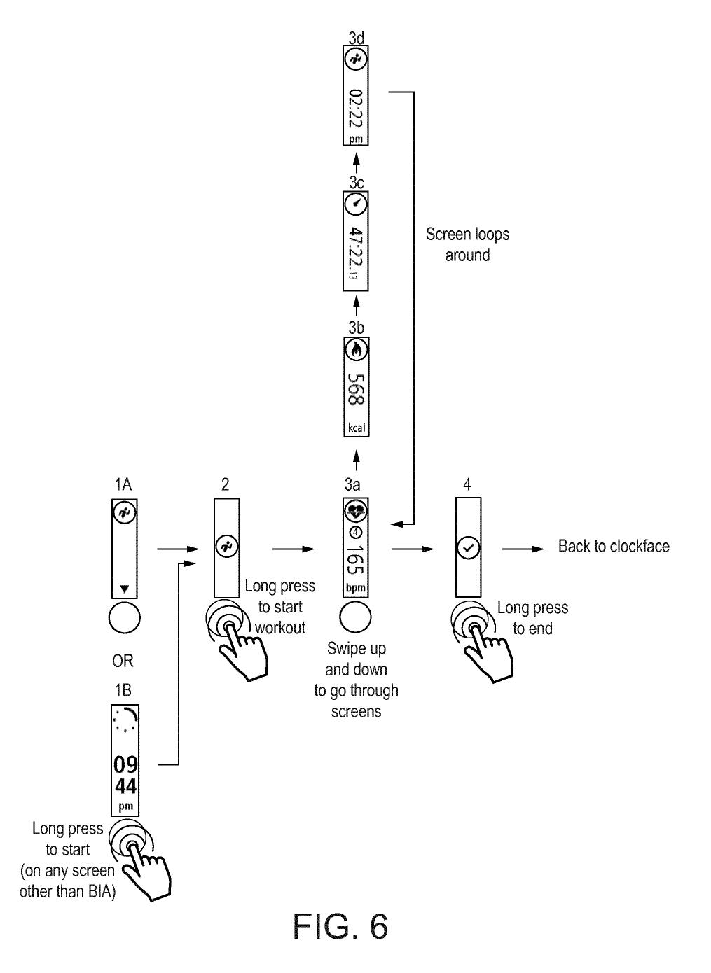

In embodiments, the tracker module 2 incorporates a processor 202 (as shown in FIG. 2). In the preferred embodiment the tracker module incorporates sensor means, described further below, which obtain body signals or measurements from which a body parameter can be calculated and these are then smoothed by the method of the invention, in this embodiment in the same processor. In the embodiment shown and described, the actual indication of the body composition parameter generated by the device is transmitted to another device for display, analysis, etc rather than being displayed on the display 4 of the activity tracker; the activity tracker can, however, provide an indication that a measurement has been completed e.g. by means of a tick icon (as shown in FIG. 5) or that the process has failed (e.g. by a cross icon on the display).

In this embodiment, the sensor means is provided on the device and is in the form of a pair of voltage/current sensors or electrodes 50, 51. One electrode 50 is on the inside of the device so that it comes into contact with the wearer's wrist in use. The other electrode 51 is on the outer-facing side of the tracker. To complete a loop between the two electrodes and through the wearer's body for measuring body parameters, the user places a finger on the outer electrode 50. A measuring current then flows from one electrode to the other through the wearer's body to measure a body parameter such as, in the embodiment described, impedance. Electrodes 50 and 51 are usually, in fact, electrode pairs each comprising an input electrode and an output electrode. A measure of body impedance is obtained as is known in the art; see, for example, US 2016/0089053 A1.

As described above, impedance can be measured using a two-point or a four-point system. If four electrodes are used, these may be provide as two pairs of side-by-side electrodes or, as shown, as two pairs of concentric electrodes. In one example, even where four electrodes are provided, one electrode on each side of the device (FIGS. 50, 51, 52 and 53) is used to determine impedance. The other electrodes 51 and 53 may be part of a feedback system, e.g. to take account of component losses in the system and provide a more accurate reading. In other embodiments, all four electrodes 50, 51, 52 and 53 are used in a four-point measuring system.

Based on the impedance measurement and using other user-specific inputs such as weight, age, height, gender a body composition parameter is calculated preferably using known BIA algorithms. The body composition parameter may be percentage fat, percentage muscle, the amount of fluid/water in the body, muscle strength.

FIG. 2 shows an example of the processing capabilities of a fitness tracker. The tracker module 2 includes a processor 202 which communicates with various function modules including input device 212, output device 214, I/O port 216, a display module 210, memory 220, GPS module 204, power supply 218, transmitter/ receiver 206, BIA module 230 and smoothing module 240. Of course, activity trackers or other wearable devices may have more or fewer functions.

As mentioned above, wearable devices with such finger contact sensors are known. Typically, these are dedicated sensors for the measurement circuit/process, e.g. for measuring body impedance or some other body parameter. Usually, these are incorporated in a device having other functions which the user can access/operate via a program menu/menus, using buttons and the device display. The menu is typically navigated, and functions are selected, input provided, etc. by means of a hard button and/or a touch screen feature of the display. In accordance with the invention, the finger electrode is modified to provide additional functions relating to the other functions of the device besides the body parameter sensing. For example, the electrode can operate as a user input means to the device processor 202 to e.g. wake up the device, start and stop workout activities, etc.

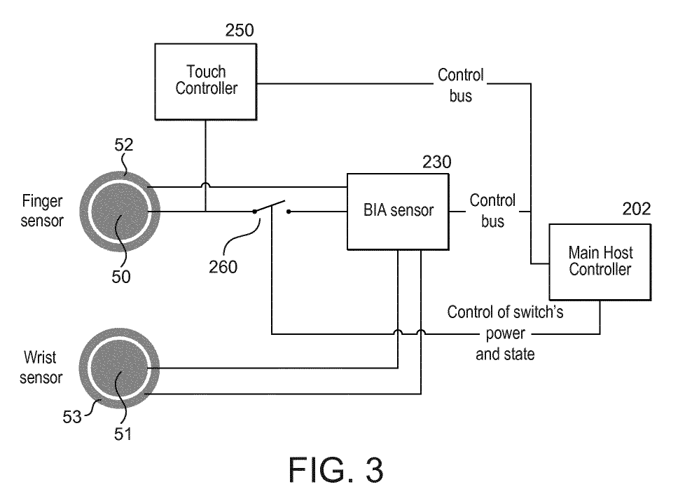

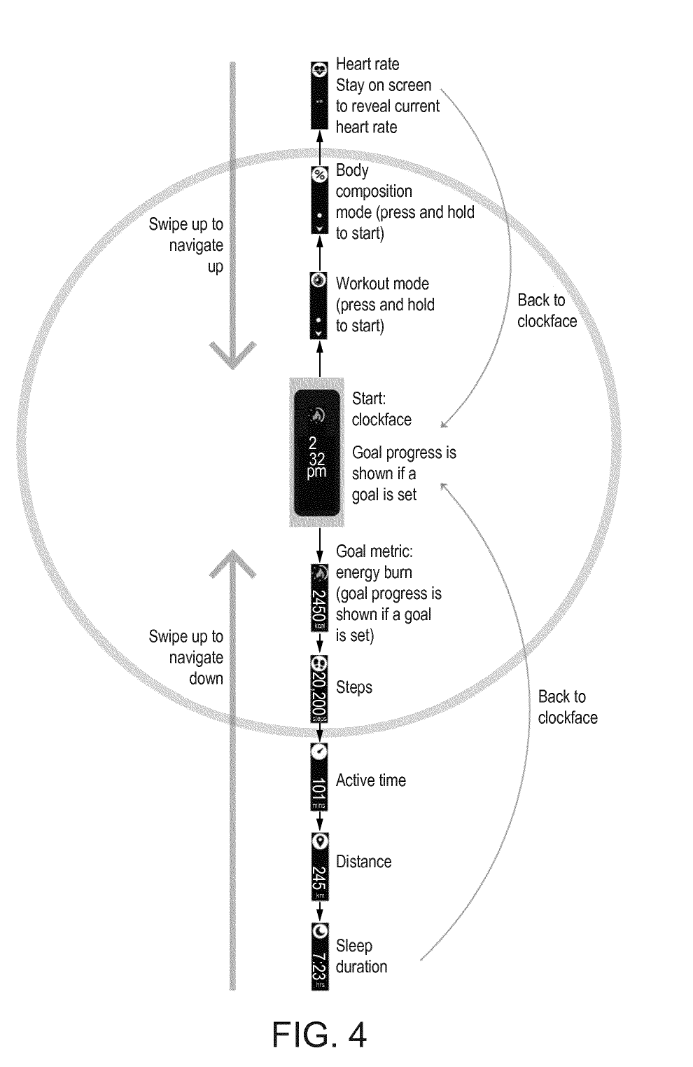

As shown in FIG. 3, the first sensor/electrode 51 or electrode pair 51, 53 previously described (see FIG. 1B) contacts the wearer's body (e.g. wrist) and is electrically connected to the measurement circuitry, e.g. BIA module 230. In a first mode, the second sensor/electrode 50 or electrode pair 50, 52 is also electrically connected to the BIA module 230 or other measurement circuit, as is known. The second sensor/electrode(s) 50 is further capable of operating in a second mode as a touch input to the processor 202 to control other functions of the device. The second electrode 50, or in the case of an electrode pair, one of the pair—here the inner second electrode 50—is also connected to a touch controller 250 which conveys input from the second electrode to the device processor 202 via a control bus. A switch 260 is provided to switch between the modes of the second electrode.

In the example shown, the default mode of the sensor is as a touch sensor/input to the device processor and the switch 260 is provided between the sensor and the BIA module 230. In the default mode, the switch 260 is open. When the user selects the BIA/measurement mode, via a menu, e.g. by swiping through mode options on the display, the switch 260 is closed and the electrode 5b operates as a measurement electrode for the BIA. After a measurement has been taken, or after the user changes the device mode, the switch 260 opens and the electrode reverts to its function as a touch input sensor. In other embodiments, the default mode could be the measurement/BIA mode, in which case the switch 260 could be in the line between the electrode 5b and the touch controller 250, or the switch could be closed in its default position.

When operating as a touch sensor, the electrode can replace or supplement the typical hard button to control/select functions such as workout modes, setting goals, switching between displays, to start or stop an activity or workout, to wake up the device or other such functions typically performed by a hard button on known devices.

It will be appreciated that whilst various aspects and embodiments of the present invention have been described, the scope of the present invention is not limited to the described embodiments but, rather, is defined by the claims.

1. A wearable device configured to perform a plurality of functions including a first function to measure a body composition parameter of a user wearing the device and one or more second functions requiring an input from the user, the device comprising:
a first electrode on an inner surface of the device arranged, when the device is worn by a user, to contact the body of the user;
a second electrode on an outer surface of the device arranged to be touched by the user;
a touch controller arranged to detect when a user touches the second electrode;
a body composition parameter measurement device arranged, when the user is in contact with the second electrode, to measure a body impedance of the user by passing a current between the first and second electrodes and detecting a voltage generated between the first and second electrode in response to the current, and to use the measured body impedance to determine a value of a body composition parameter;
a processor arranged to control a switch that, when in a first state, electrically connects the second electrode with the touch controller, such that a detected touch by the user on the second electrode can be used as an input to control a second function of the device, and, when in a second state, electrically connects the second electrode electrode with the body composition parameter measurement device; and
an input device arranged to receive an input from the user to select one of the plurality of functions, and to provide an indication of said selected function to the processor,
wherein the processor is further arranged to change the state of the switch from the first state to the second state when the input device indicates that the selected function is the first function.

2. A wearable device as claimed in claim 1, wherein one or more second functions of the device that require an input from the user can comprise one or more of the following: to wake up the device from a low power state, e.g. by activating the input device, e.g. touch screen display device; to start and/or stop a timer or stop watch; and to indicate the beginning and/or end of an activity.

3. A wearable device as claimed in claim 1, wherein the plurality of functions which the device is arranged to perform comprises one or more third functions that do not require input from the user.

4. A wearable device as claimed in claim 1, wherein the plurality of functions which the device is arranged to perform comprises one or more third functions, the one or more third functions being one or more of providing a current heart rate of the user, providing the current time to the user and providing a set of metrics associated with a completed activity.

5. A wearable device as claimed in claim 1, wherein the body composition parameter is at least one of body fat percentage and body muscle percentage.

6. A wearable device as claimed in claim 1, wherein the device is arranged to be worn on a wrist of the user, such that the first electrode is configured to be in contact with the wrist, and the second electrode is configured to come into contact with a finger of the user.

7. A wearable device as claimed claim 1, wherein the second electrode has a convex surface.
